(19) United States
(12) Patent Application Publication
(10) Pub. No.: US 2007/0092962 A1
(43) Pub. Date: Apr. 26, 2007

(54) Carbon Neutralization System (CNS) for CO2 sequestering

(75) Inventor: Norman J. Sheppard, Dhahran (SA)

Correspondence Address: BRACEWELL & GIULIANI LLP, P.O. BOX 61389, HOUSTON, TX, 77208-1389, US

(73) Assignee: Saudi Arabian Oil Company, Dhahran (SA)

(21) Appl. No.: 11/546,104

(22) Filed: Oct. 11, 2006

Related U.S. Application Data
(60) Provisional application No. 60/728,541, filed on Oct. 20, 2005.

(51) Int. Cl. A61L 9/01; C12M 1/00; C12N 1/12; A01G 7/00

(52) U.S. Cl. 435/266; 435/257.1; 435/292.1; 47/1.4

(57) ABSTRACT

A device and method for carbon dioxide sequestering involving the use of a photo-bioreactor with Light Emitting Diodes (LED's) for the cost-effective photo-fixation of carbon dioxide (CO₂). This device and method is useful for removing undesirable carbon dioxide from waste streams.

### RELATED APPLICATIONS

This application is related to and claims priority and benefit of U.S. Provisional Patent Application Ser. No. 60/728,541, filed Oct. 20, 2005, titled “Carbon Neutralization System (CNS) for CO₂ Sequestering,” which is incorporated herein by reference in its entirety.

### BACKGROUND OF THE INVENTION

1. Field of the Invention

This invention generally relates to the field of carbon dioxide sequestering. More specifically, the present invention relates to a photo-bioreactor with pulsing Light Emitting Diodes (LED's) for the cost-effective photo-fixation of carbon dioxide (CO₂).

2. Description of the Related Art

Power stations burn fossil hydrocarbons such as coal, oil, and gas in order to meet the world's rampant demand for affordable energy. The combustion of these hydrocarbons releases large amounts of carbon dioxide into the atmosphere. For example, the combustion of a petroleum fuel such as liquid paraffinic oil produces up to three tons of carbon dioxide for every one ton of liquid hydrocarbon. As discussed below, the carbon dioxide emissions will potentially, in a time scale of about 10 to 80 years, become a limiting factor in the use of hydrocarbon fuels.

Carbon dioxide is a “greenhouse gas” that blankets the earth and traps heat inside the atmosphere. As the burning of hydrocarbons increases the concentration of carbon dioxide, it is generally accepted that the atmosphere's “greenhouse effect” will be unnaturally enhanced. As a result, increasing amounts of heat will be trapped near the earth instead of escaping into space. This phenomenon, known as global warming, could result in a number of consequences. These include higher average global temperatures, unpredictable weather, the melting of Antarctic, Arctic and glacial ice sheets, rising sea levels, and loss of wildlife.

In addition to environmental restraints, legal and administrative regulations restrict the release of carbon dioxide, and hence the burning of hydrocarbons. Partly as a result there is now a European market for trade in carbon dioxide emissions permits, typically valued in mid 2005 at about $35/metric tonne of carbon dioxide. Furthermore, there are potential legal actions that could be brought against some of the users of hydrocarbons, and by implication their hydrocarbon suppliers, on the grounds that they “knowingly and willfully” emit substances that are damaging to the environment. Given these restraints, there exists a clear need to mitigate the emission of carbon dioxide.

Technology offers a range of solutions for dealing with the increase in greenhouse gas emissions associated with rampant energy demand. For example, wind and solar power stations are able to produce electricity without emitting carbon dioxide. Despite the existence of these technologies, there are currently no medium-term, fully viable alternatives to the burning of fossil fuels. Therefore, it would be advantageous to develop a new method and apparatus that allows the cost effective sequestration of carbon dioxide from sources that burn hydrocarbons. This would allow for a more tolerable climate as well as continued burning of the Earth's considerable reserves of fossil fuels.

It is noted that in nature, plant life sequesters CO₂ through a process known as photo-synthesis. The earliest known global adopters of this process, cyano-bacteria, are still the most prolific converters of CO₂, having spent about 3.5 billion years perfecting carbon sequestering. In inoculated slime ponds, these bacteria can convert between 10 and 30 grams of carbon per square meter per day, depending on the availability of nutrients (primarily nitrates and phosphates), temperature, insolation (sun) intensity, mixing conditions, competing organisms, and similar factors.

While natural slime ponds are able to sequester CO₂, there are numerous problems associated with using this technique in a hydrocarbon fired power station. Sunlit ponds are only able to sequester CO₂ during certain times of the day, while power plants often operate continuously. Furthermore, the rate of CO₂ sequestration remains dependent on natural variations in cloud cover, rainfall, and night fall.

Attempts have been made to culture algae in an artificial environment. For example:

Patent No. DE-10222214 teaches a two-chamber, vertical bioreactor design. The light source is continuous, such as a neon tube light or incandescent bulb. However, the light source is not controllable, emits from the wrong spectrum for preferred CO₂ sequestration and wastes energy.

Patent No. JP-2002315569 discusses light sources used to industrially culture a large amount of algae at a constant rate of proliferation, 24 hours a day, and during all times of year. The proliferation of algae is promoted by irradiating the culture with monochromatic light having a wave length exhibiting > or =60 specific absorbance by chlorophyll (a). The light is emitted by a non-flashing LED, and the wavelength of the light is 630-690 nm (red light) and/or 400-460 nm (blue or violet light). The photosynthesis of carotenoid can be promoted in the algae-culturing liquid by the irradiation with 400-500 nm monochromatic light (violet, blue, and green), and the photosynthesis of phycocyanin can be promoted by the irradiation with 500-630 nm monochromatic light (green, yellow, and orange). The patent does not, however, teach intermittent LED lighting, nor does it teach delivering the 660 nm light required by chlorophyll to optimize carbon dioxide sequestration.

Patent No. WO-02099032 discusses a device and method for cultivating micro algae in a parabolic-shaped container. Productivity is realized through sufficient stirring of the culture solution by blowing gas into the culture container. The micro algae are prevented from adhering to the wall surface and/or precipitating onto the bottom surface in order to maintain a high culture efficiency. The patent teaches, however, that cultivation occurs via natural sunlight.

Patent No. WO-9519424 discusses a device for cultivating algae in a cylindrical bioreactor. This patent also teaches cultivation using natural sunlight.

U.S. Pat. No. 3,986,297 discusses a sealed double tank assembly for use in artificially cultivating photosynthetic substances. This sealed double tank assembly comprises an inner tank containing culture fluid and an outer tank in which water is circulated for temperature control. The inner tank is provided with a plurality of nozzles which emit nutrients such as mixed gases of carbon dioxide and ammonia, one or more sources of light substantially similar to natural light for intermittent light application to the interior of the tank assembly, and agitator vanes to agitate the culture fluid. The patent does not, however, teach a preferred source for intermittent light. Also, the agitator vanes would damage the cell walls, which would cause cell death.

Other existing photo-bioreactor designs have attempted to shorten light/dark cycle times by pumping an aquatic culture through a lighted zone at increased speeds. This approach is limited, however, by the turbulent flow of the fluid, the hydraulic power requirement and by cell death due to cell wall rupture under hydrodynamic stress.

It would be advantageous to develop a new method and apparatus that maximizes the rate of carbon dioxide sequestration while simultaneously minimizing the energy required to operate the system. It would also be advantageous to control the light-dark cycle to promote the maximum photosynthetic rate of the algae. It would be advantageous to avoid photo-inhibition due to excessive light levels. It would also be advantageous to use light of the very narrow wavelength range (660 nm+/−10 nm) required by the chlorophyll in the algae. Furthermore, it would advantageous for the path of the light to be short so as to minimize the mutual shading i.e. occulation of the algae. It would also be advantageous to provide a continuous process that can be used for both industrial-scale processes and smaller lab or batch-scale processes. It would be advantageous to provide a process that can be cleaned continuously to counteract fouling by the algae. Finally, it would be advantageous to use very little energy pumping the suspended algae culture around the system.

### SUMMARY OF THE INVENTION

The current invention includes a continuous process photo-bioreactor and method of operating said bioreactor using flashing light emitting diodes (“LED's”) to artificially force and accelerate chlorophyll based photosynthesis in blue green algae (cyano-bacteria) and other related, uni-cellular organisms, either naturally occurring, derived there from, manipulated/created by artificial means or otherwise cultivated. The LED's preferably emit light tightly centered on a wavelength of 660 nm to optimize carbon dioxide sequestration while minimizing energy costs. The sequestered carbon dioxide can emanate from, for example, flue gas stacks from large stationary sources such as power plants, cement works and the like that burn solid, liquid or gaseous hydrocarbons.

The increase in carbon dioxide sequestration is realized by accelerating and compressing the natural, day-night diurnal cycle to a fraction of its natural cycle time, preferably milliseconds, by flashing the LEDs. The minimization of the energy costs is achieved by electronically linking the LED's light cycle and light fraction time to the oxygen content and other measures of the culture (oxygen output being a measure of photosynthetic activity), so that the culture can be automatically kept at or near the maximum photosynthetic rate for the prevailing conditions of nutrients, gas flow, CO₂ concentration, and the like. To achieve these results, it is anticipated that the “flash” will preferably last in the order of pico to micro seconds, and that the subsequent dark period will be in the approximate range of milli seconds up to about one second.

The process occurs in a closed, “forced” environment in order to increase the rate of CO₂ conversion and achieve consistent, quantifiable, certifiable and continuous sequestration. The process would produce valuable algae that, depending on the business case and the local market economy, can be sold and used for a variety of purposes, from specialty chemicals to biomass for combustion.

The process and apparatus would minimize energy costs in a number of ways. Much of the energy conservation is achieved by using carefully controlled flashing LED's that emit light tightly centered on a wavelength of 660 nm. The photon (light) stream from the LED's is intermittent (i.e. broken up), which saves energy as compared to continuous lighting. The LED's preferably emit light only at the very narrow wavelength required by the chlorophyll in the algae, which increases the energy efficiency of the system. Furthermore, the light-dark cycle times are controlled so as to match the overall photosynthesis cycle (PS I+PS II+the dark [Calvin] cycle), thus maximizing carbon dioxide uptake. Phyto-inhibition, which occurs at excessive light levels, is largely avoided, and the algae are photo-acclimated to grow in low average light levels.

The bioreactor apparatus of the present invention preferably includes a sealed housing or building having a series of stacked, generally parallel trays running there through, each of wide rectangular profile. They are preferably inclined slightly from the horizontal. Energy savings are achieved by arranging the bioreactor horizontally, rather like the tubes in a contemporary, steam-raising boiler. This orientation saves energy because it obviates most of the mass flow and control problems that are seen in vertically arranged bio-reactors. Very little energy is expended in pumping a culture through a horizontal system, since most of the flow is achieved by orienting the horizontal bioreactor slightly downhill. As a result, most of the flow is due to gravity, with the remainder being achieved by the flowing of the gas. Due to the horizontal orientation, only minimal energy is needed to run the pump to return the aquatic culture to the top end of the bioreactor. For instance, a low-pressure, “Archimedes Screw” pump can be used to gently return the aquatic culture to the top end of the reactor using very little energy. The water cycle within the system is almost closed, only requiring makeup water to compensate for some evaporation through the gas vents, and some water entrained with the harvested algae.

As a further advantage, because the reactor trays are modular, and hence stacked, the system is easily scaled, for example, from a 16 MW gas turbine right through a huge 16,000 MW power station. Such modularity of construction, and hence scalability, has not been seen in other photo-bioreactors. Further, the modular design allows different strains of bacteria to be grown in different groups of trays, if desired.

Finally, as the reactor trays are horizontal and modular, maintenance of the trays will be easy. For major overhauls, the trays can be pulled from the assembly, rather like the current procedure for cleaning and de-scaling boiler tubes. For intermittent, in-situ cleaning, which is important to avoid algal fouling of the LED's radiant surfaces, a low-power red laser can be swung to the ends of the temporarily emptied tray channel. The laser will quickly scan the interior of the channels in a pre-set pattern, thus burning off any organic residues without scratching the polycarbonate, optical surfaces. For longer tray lengths, a small, trolley-mounted red laser can be pulled through the tray while rotating rapidly in order to achieve de-fouling. It is known that live Chlamydomonas Reinhardtii cells can be made to rotate while pinned in a laser “trap.” The energy of the light beam can thus be used to manipulate and trap cells much like the way that the wind moves objects of a larger scale. Hence the laser approach described above can also be effective as a cleaning device in association with the present invention.

### BRIEF DESCRIPTION OF THE DRAWINGS

So that the manner in which the features, advantages and objects of the invention, as well as others which will become apparent, may be understood in more detail, more particular description of the invention briefly summarized above may be had by reference to the embodiment thereof which is illustrated in the appended drawings, which form a part of this specification. It is to be noted, however, that the drawings illustrate only a preferred embodiment of the invention and is therefore not to be considered limiting of the invention's scope as it may admit to other equally effective embodiments.

FIG. 1 shows a graph illustrating the relationship between algae conversion rates and light cycle time.

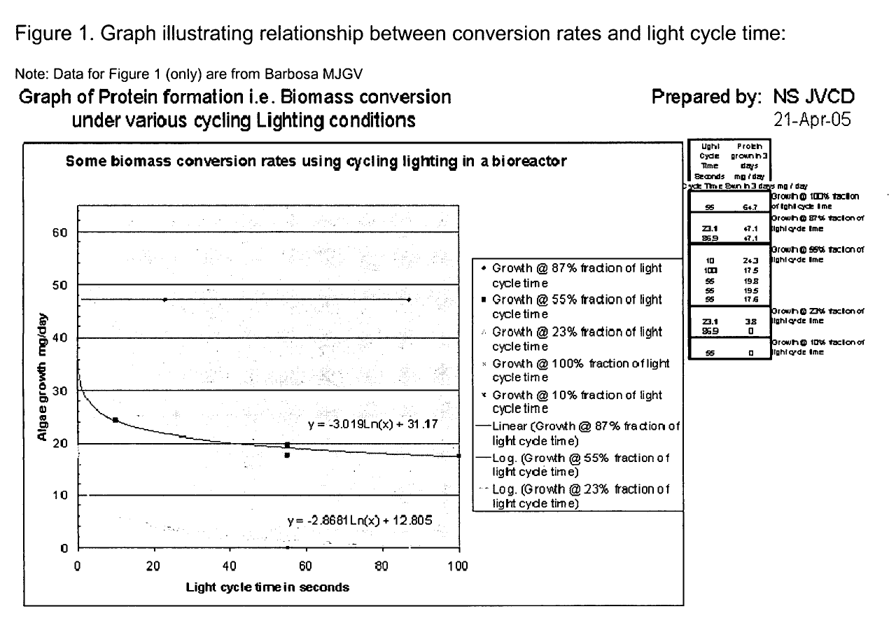

FIG. 2 shows a front view of trays, mounting and housing in a CNS building according to an embodiment of the present invention.

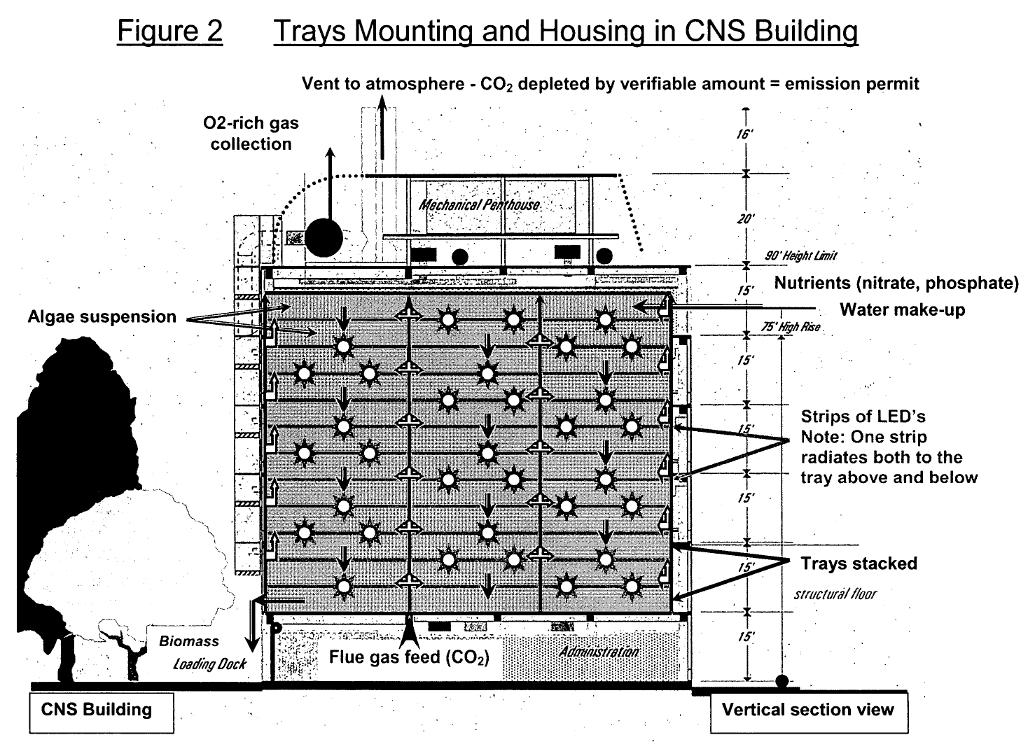

FIG. 3 shows a perspective view of trays with LED's attached thereto according to an embodiment of the present invention.

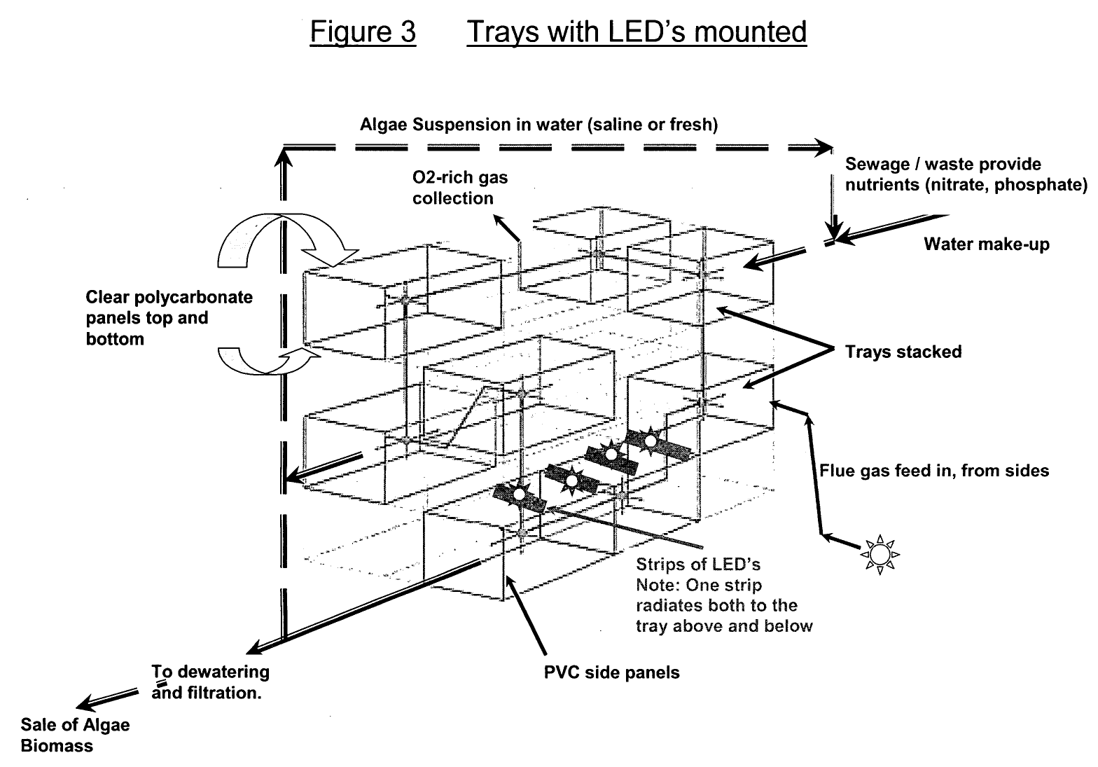

FIG. 4 shows a process for on-line bio-fixation of CO₂ from flue stack gases of coal, oil and gas-fired power stations according to an embodiment of the present invention.

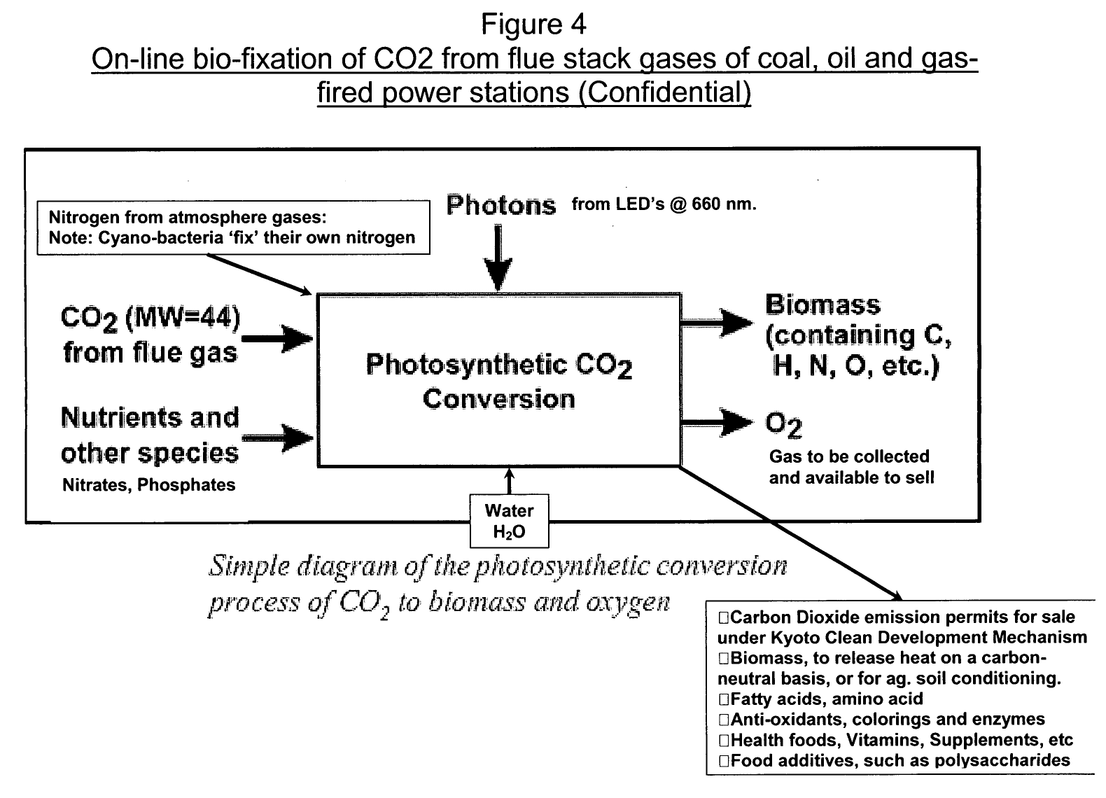

### DETAILED DESCRIPTION

The present invention includes a method and device for optimizing the sequestration of carbon dioxide while minimizing the energy costs. In a preferred embodiment, the device provides for the forced, artificially driven photosynthesis of cyano-bacteria (blue green algae) by the use of pulsed or flashing light emitting diodes as an artificial light source.

The device for the photofixation of CO₂ of the present invention includes an outer wall defining a containment area. A CO₂-rich gas source operable to provide CO₂-rich gas into the containment area is also provided. A plurality of trays is housed within the containment area, the trays having a bottom and two sides. The trays are operable to circulate an aquatic culture of a photosynthetic organism operable to convert CO₂-rich gas to O₂-rich gas. The aquatic culture is circulated within the trays, that is, that the aquatic culture is moved generally down the tray while generally being maintained between the two sides. The trays define holes with an original average diameter. The holes allow for the passage of the CO₂-rich gas through the trays when exposed to the CO₂-rich gas. An artificial light source is provided that is operable to radiate the aquatic culture. The artificial light source is able to deliver intermittent flashes of light centered on a preselected wavelength range, the intermittent flashes being deliverable with a predetermined frequency and duration of light and a predetermined period wherein the artificial light source does not emit light. Thus, periods of light exposure and removal of light can be pre-determined. The device also includes an exhaust line operable to receive the converted CO2-rich gas as an exhaust stream from the containment area. The exhaust stream is the result of the conversion of the CO₂-rich gas to a gas that has less CO₂.

In a preferred embodiment, the device also includes a conveyance apparatus in communication with the aquatic culture such that the conveyance apparatus is operable to promote circulation of the aquatic culture along the tray with laminar flow. Examples of preferred conveyance apparatus include an Archimedes' screw and a reciprocal pump for moving the aquatic culture generally down the tray.

In another preferred embodiment, a recycle line communicates between the exhaust line and the CO₂-rich gas source such that at least a portion of the exhaust stream is directed into communication with the CO₂-rich gas source for introduction into the containment area. This allows further processing of at least part of the exhaust line for further removal of CO₂. The recycle line can communicate in manners generally known in the art, including being mixed with the CO₂-rich gas source prior to addition to the containment area or being added in a separate line directly into the containment area.

In a particularly preferred embodiment, the trays also include a top. The top, in conjunction with the bottom and sides define an enclosed tray area. The enclosed tray area can be controlled separately from the containment area. In one preferred embodiment, a gas pressure differential is controlled between the enclosed tray area and the containment area. Control is preferably achieved through the use of a pressure regulator.

In a further preferred embodiment, the artificial light source is embedded in the top of the tray. In alternate embodiments, the artificial light source is embedded in at least on of the sides or the bottom of the tray. In a particularly preferred embodiment, the top of one tray acts as the bottom of the tray above it and the light source is embedded therein. The trays generally define a rectangular cross-section and the tray bottoms are inclined from horizontal such that flow of the aquatic culture through the trays is enhanced by gravity. Another preferred embodiment includes a generally trapezoidal cross-section of the trays. The trays are preferably made of a transparent material on at least the bottom or one side, such that light from the artificial light source generally passes through the transparent material and is capable of radiating the aquatic culture in this manner. An exemplary transparent material includes polycarbonate.

The preferred artificial light source of the invention is operable to deliver intermittent flashes of light in the wavelength range 660nm+/−10 nm. It is preferable to deliver the intermittent flashes with a frequency of less than two seconds and more preferably, one second. It is also preferable to deliver the intermittent flashes with a duration of less than about 0.5 seconds or more preferably 0.1 seconds.

The aquatic culture preferably flows at a flow rate through the trays of generally less than two meters per second. This reduces hydrodynamic stresses. A filter in communication with the aquatic culture is included in one embodiment for removing at least a portion of the photosynthetic organism from the tray. These photosynthetic organisms can be harvested for various uses. A preferred variety of the photosynthetic organism includes cyanobacteria, with blue-green algae being a particularly preferred variety.

The trays of the invention can also include a non-stick material on at least a portion of an inner surface that is in communication with the photosynthetic organism. The non-stick material is selected to minimize adhesion between the non-stick material and the photosynthetic organism, thus avoiding sticking and clumping. A preferred non-stick material includes polytetrafluoroethlyene.

By maintaining the original average diameter of the holes of the trays smaller that an average diameter of the photosynthetic organism in a preferred embodiment, seepage of the photosynthetic organism through the holes of the tray is discouraged.

For additional light capability, the outer wall includes or is equipped with movable shades, such that opening the shades allows natural sunlight to radiate the aquatic culture.

In this manner, the device of claim provides a renewable manner of removing carbon dioxide from a gas source, including from a waste gas from an industrial process.

The invention also includes a method of photofixation of CO₂. This includes the steps of circulating an aquatic culture having photosynthetic cyanobacteria on a plurality of trays, the trays preferably being in communication with one another. The trays, having a bottom and sides, are contained within the containment area. The method includes providing the CO2-rich source gas into the containment area such that the CO2-rich source gas contacts the aquatic culture and irradiating the aquatic culture through the use of artificial light source. The artificial light source has a plurality of light emitting diodes operable to deliver intermittent flashes of light deliverable with a predetermined frequency and duration of light and a predetermined period wherein the artificial light source does not emit light. This method includes the sequestering of a portion of the carbon from the CO₂-rich source gas within the cyanobacteria through the process of photosynthesis. An exhaust stream is thereby produced having a reduced quantity of CO₂ as compared to the CO2-rich source gas. The exhaust stream is removed from the containment area. The sequestering of a portion of the carbon from the CO₂-rich source gas is accomplished by contacting the CO₂-rich source gas with the cyanobacteria. In a preferred embodiment, this is accomplished when the bottom of the trays have plurality of holes such that the CO₂-rich source passing through the holes contacts the aquatic culture, the holes having an original average diameter selected in view of the average diameter of the cyanobacteria. In a preferred embodiment, the method also includes promoting circulation of the aquatic culture using a conveyance apparatus, the conveyance apparatus being in communication with the aquatic culture such that the aquatic culture moves with laminar flow. Preferred embodiments of conveyance include the use of Archimedes' screw or reciprocal pump. The method also includes placing the trays at an incline to the horizontal wherein circulation of the aquatic culture through the trays is enhanced by gravity. The method can include the step of filtering at least a portion of the cyanobacteria on the tray.

A preferred embodiment of the method includes the step of recycling at least a portion of the exhaust gas back into the containment area for further contact with the aquatic culture. The exhaust stream includes an increase in the O₂ produced by the photosynthetic cyanobacteria in the exhaust stream. The method of the invention includes an embodiment where the O₂ is captured.

The method includes in a preferred embodiment providing a top for the tray that together with the bottom and sides defines an enclosed tray. The gas pressure differential between the enclosed tray area and the containment area is controlled preferably through the use of a gas pressure regulator. The step of controlling the pressure within the containment area of the CO₂-rich source gas on the tray bottoms discourages the aqueous culture from flowing through holes defined by the trays. The preferred embodiment of the method includes regulating the pressure wherein the gas velocity through the holes defined by the trays of the CO2-rich source gas is less than about 40 meters per second.

The method includes an additional optional step of irradiating the aquatic culture with natural sunlight.

The bioreactor apparatus of the present invention preferably includes a sealed housing defined by the outer wall or a building having a series of stacked, parallel trays positioned therein, each of wide rectangular profile and inclined slightly to the horizontal. Each bioreactor tray preferably has two plastic, e.g., PVC sides to form the sides of the tray, as well as two transparent sides to form the top and bottom of the tray. In the maximum preferred embodiment, the ‘footprint’ of the reactor is up to 1000 meters long and up to 1000 meters wide and about one kilometer long, and the sides of the trays are about one centimeter tall. However, the dimensions of the reactor can be varied to a great degree, for example, according to whether the design is being utilized in industry or in a scaled down bench top model. Each tray could also be lined with a non-stick coating. In one embodiment, the non-stick coating is PTFE. The top and/or bottom surfaces of the trays, i.e., the transparent cover, consist of an optically transparent material such as polycarbonate (e.g. Lexan). These top and bottom surfaces are bonded to the plastic sides of the tray. Rows of LEDs are embedded within the top transparent surface. The outside surfaces of the bioreactor may be provided with movable shades, allowing sunlight to supply additional energy to fuel sequestration.

Pre-treated and cleaned flue gases are admitting to the trays via micron size holes (perforations) drilled in the plastic side walls. These perforations will preferably have an internal diameter that is less than the average diameter of the algae cells in the culture. Gas pressure will prevent the aqueous culture flowing back through the perforations.

The number of perforations in the side walls is determined by the gas pressure so that the gas velocity up through a perforation does not exceed approximately 40 meters per second (˜80 mph), thus limiting cell death due to hydro dynamic stress. The gas pressure differential between the inside and the outside of the trays is regulated using a standard pressure regulator.

Each tray carries a water-based nutrient medium inoculated with a culture of cyanobacteria. In the preferred embodiment, the cyanobacteria selected for the culture is acclimated to live and photosynthesize under low light intensities. The gas flowing through the channel bubbles through the liquid medium. After having passed through the liquid medium, the gas is collected at the exit point and recycled back for CO₂ enrichment by admixing the scrubbed, CO₂ source gas.

Preferably, the transparent surfaces of the trays are embedded at regular intervals with one or more commercially available, low power, high efficiency, flat-plate LEDs that emit reddish-yellow light tightly centered on a wavelength of approximately 660 nm (reddish-yellow). The LEDs can be positioned adjacent to, as opposed to on, the trays, if desired. Also, one LED can be used to provide light to multiple trays. All photosynthesis can be achieved using solely the LED's, if desired, and natural sunlight is not required to operate the apparatus. As a result, operation can continue through the night, if desired, and is independent of ambient weather conditions. In one embodiment, the walls and roof of the structure can be formed of a transparent material, for example plastic, so that the additional energy from 643-660 nm sunlight can penetrate the walls and provide additional incident energy.

In the preferred embodiment, wherein the reactor ‘footprint’ is between 1000 [10³] square meters and 1,000,000 [10⁶] square meters (a square kilometer), each LED is 5 millimeters square, and LED's are fixed uniformly over the length of the reactor trays, about 1.5-1.6 meters apart. Each ton of hydrocarbon burned produces about three tons of carbon dioxide, which at 83,000 MT of fuel per day equates to about 250,000 MT of CO₂ produced per day. The carbon sequestration rate according to the present invention is preferably up to 60 grams of carbon per square meter per day. In a preferred embodiment, the building is approximately one kilometer square and 15-30 meters high, with approximately 1500 trays closely and vertically spaced at approximately 1 cm apart or less. The water based medium and suspended algae preferably passes through the structure at about 1 meter per second, for a total transit time of about 15 minutes.

All conditions in the water-based medium and gas are preferably tightly and automatically controlled and monitored. These conditions include temperature (preferably between 25 and 40 C), nutrient levels (primarily of nitrates and phosphates), acidity (pH), oxygen content, CO₂ content, and gas flow rates. Before entering the bioreactor, the flue gases are pre-cleaned of NOx and SOx in a water-scrubber and pre-blended with ambient atmosphere to control the CO₂ content according to the optimum level for the cyano-bacteria photosynthesis. The preferred CO₂ content is between 8 and 10% by volume.

In a preferred embodiment, all of the conditions are tightly controlled, with on-line monitoring and automatic compensation for drift from the optimum conditions. Trays that go off spec, for example due to biological contamination or blockage, will preferably be temporarily closed and flushed through with a water-based cleaning solution and a gantry mounted traveling raking system.

The reactor tray is set at a slight angle so that when the water based culture medium with suspended cyano-bacteria is pumped into one end, the medium will flow the length of the tray under the force of gravity at about one meter per second. Once the medium reaches the end of the tray, a fraction of the cyano-bacteria will be filtered off while the balance of the medium is recycled back to the beginning of each tray. The filtered cyano-bacteria can then be used, for example, as cattle feed, nutritional supplements, or even as bio-mass fuel for burning.

Suitable digital control systems, instrumentation, and software are used to control the reactor conditions. They can be acquired, for example, from APPLIKON of the Netherlands and USA. Suitable LEDs are also commercially available. For example, the SHARP LED Type GL8 TR22 produces 660 nm light, which is near the optimum for the photo-sensitive compounds present in cyano-bacteria.

To use these devices and methods on an industrial scale, the shallow, one-cm high trays can be easily stacked. In a large Carbon Neutralization System (CNS), a sealed building could contain a series of stacked, parallel reactor trays, each of a wide, rectangular profile and inclined slightly to the horizontal.

Preferably, the present invention can be utilized in a hydrocarbon burning industrial power station of capacity between about 16 and 16,000 MW. TABLE 1 shows the relevant approximate data for facilities at or near both ends of the aforementioned range. As indicated by the data, there is preferably a linear relationship between the upper and lower data ranges:

TABLE 1
CNS DATA FOR PILOT SCALE/LARGE SCALE FACILITIES

| | 16 MW (pilot plant) | 16,000 MW |
|---|---|---|
| Type of fuel | Middle distillate or | Medium or Heavy Fuel |
| | Light Fuel Oil | Oil |
| Fuel Use per day | 100 Thousand liters | 100 Million Liters |
| Type of equipment | 1× Gas Turbine | Steam raising boilers |
| Daily Performance | Approx. 40% efficiency | Approx. 35% efficiency |
| Electric Power - | 16 MW | 16,000 MW |
| Gross Produced | | |
| Electric Power - | 14 MW | 14,000 MW |
| Net Produced | | |
| Total No. of LEDs | 90 Million | 90 Billion |
| Building size | 50 × 50 × 6 meters | 1 km sq. by 15 meter |
| | | high |
| Tray height | 1 cm | 1 cm |
| Algae bio mass | Approx. 80 MT | Approx. 80,000 MT/day |
| (dry) | output/day | |
| Examples of | Spirulina, chlorella, | Spirulina, chlorella, |
| type of algae | etc . . . | etc . . . |
| Quantity CO₂ | 250 MT/day | 250,000 MT/day |
| captured | | |

The device and method of the present invention can be utilized in a variety of industries and by a variety of types of companies, including but not limited to additive and specialty chemical companies, pharmaceutical and cosmetics companies, functional food companies, farmers, and generators of carbon dioxide. Further, in embodiments of the present invention, warm water can be extracted from the device's biostatic, temperature controlled heat exchangers and used in, for example, providing irrigation water and/or an external heating scheme, and output from external sewage farms can be utilized to formulate and make up the water-based nutrient medium for the cyano-bacteria, in the process removing undesirable phosphates and nitrates from the sewage farm's effluent discharge. Finally, oxygen-rich air can be captured at the gas vents, and offered for sale, if found to be economically viable.

While the invention has been shown or described in only some of its forms, it should be apparent to those skilled in the art that it is not so limited, but is susceptible to various changes without departing from the scope of the invention. For example, the cycling of the LED may be used in vertical applications. Recycle streams and other process tools are also encompassed within this invention.

What is claimed is:

1. A device for the photofixation of CO₂, the device comprising:
an outer wall, the outer wall defining a containment area;
a CO₂-rich gas source operable to provide CO₂-rich gas into the containment area;
a plurality of trays housed within the containment area, the trays having a bottom and two sides, the trays being operable to circulate within the trays an aquatic culture of a photosynthetic organism operable to convert CO₂-rich gas to O₂-rich gas, the trays defining holes having an original average diameter, the holes operable to allow for the passage of the CO₂-rich gas through the trays;
an artificial light source operable to radiate the aquatic culture, the artificial light source operable to deliver intermittent flashes of light centered on a preselected wavelength range, the intermittent flashes being deliverable with a predetermined frequency and duration of light and a predetermined period wherein the artificial light source does not emit light; and
an exhaust line operable to receive the converted CO2-rich gas as an exhaust stream from the containment area.

2. The device of claim 1, further comprising a conveyance apparatus, the conveyance apparatus being in communication with the aquatic culture such that the conveyance apparatus is operable to promote circulation of the aquatic culture along the trays with laminar flow.

3. The device of claim 2, wherein the conveyance apparatus comprises an Archimedes' screw.

4. The device of claim 2, wherein the conveyance apparatus comprises a reciprocal pump.

5. The device of claim 1, further comprising a recycle line in communication between the exhaust line and the CO₂-rich gas source such that at least a portion of the exhaust stream is directed into communication with the CO2-rich gas source for introduction into the containment area.

6. The device of claim 1, wherein the trays include a top.

7. The device of claim 6, wherein the top of one tray in conjunction with the bottom and sides of one tray defines an enclosed tray area.

8. The device of claim 7, further comprising the step of controlling a gas pressure differential between the enclosed tray area and the containment area

9. The device of claim 8, wherein the gas pressure differential is controlled through the use of a pressure regulator.

10. The device of claim 6, wherein the artificial light source is embedded in the top of at least one tray.

11. The device of claim 1, wherein the artificial light source is operable to deliver intermittent flashes of light in the wavelength range 660 nm+/−10 nm

12. The device of claim 1, wherein the artificial light source is operable to deliver the intermittent flashes with a frequency of less than one second and a duration of less than 0.1 seconds

13. The device of claim 1, wherein the trays generally define a rectangular cross-section and wherein the bottoms of at least a portion of the plurality of the trays are inclined from horizontal such that flow of the aquatic culture through at least a portion of the plurality of trays is enhanced by gravity.

14. The device of claim 1, wherein the trays are made of a transparent material for at least the bottom or one side, such that the light centered on a narrow wavelength passes through the transparent material.

15. The device of claim 14, wherein the transparent material comprises polycarbonate.

16. The device of claim 1, wherein the flow of the aquatic culture defines a flow rate through the trays, the flow rate being generally less than two meters per second.

17. The device of claim 1, further comprising a filter in communication with the aquatic culture, the filter being operable to remove at least a portion of the photosynthetic organism from the tray.

18. The device of claim 1, wherein the photosynthetic organism comprises a type of cyanobacteria.

19. The device of claim 18, wherein the cyanobacteria comprises blue-green algae.

20. The device of claim 1, wherein the trays further comprises a non-stick material on at least a portion of an inner surface of the tray, the inner surface being in communication with the photosynthetic organism, the non-stick material being selected to minimize adhesion between the non-stick material and the photosynthetic organism.

21. The device of claim 20, wherein the non-stick material comprises of polytetrafluoroethlyene.

22. The device of claim 1, wherein the original average diameter of the holes defined by the trays is smaller that an average diameter of the photosynthetic organism.

23. The device of claim 1, wherein the artificial light source consists of light emitting diodes.

24. The device of claim 1, wherein the artificial light source is embedded in the bottom or at least one side of the tray.

25. The device of claim 1, where the intermittent flashes are delivered with a frequency of about less than one second and a duration of about less than 0.1 seconds.

26. The device of claim 1, wherein the outer wall includes movable shades being operable to allow natural sunlight to radiate the aquatic culture.

27. The device of claim 1, wherein the CO2-rich gas source is a waste gas from an industrial process.

28. A method of photofixation of CO₂, the method comprising the steps of:
circulating of an aquatic culture comprised of a photosynthetic cyanobacteria, the aquatic culture being circulated on a plurality of trays, the trays being contained within an outer wall, the outer wall defining a containment area, the trays having a bottom and two sides;
providing CO2-rich source gas into the containment area such that the CO2-rich source gas contacts the aquatic culture;
irradiating the aquatic culture through the use of an artificial light source, the artificial light source having a plurality of light emitting diodes operable to deliver intermittent flashes of light centered on a preselected wavelength range, the intermittent flashes being deliverable with a predetermined frequency and duration of light and a predetermined period wherein the artificial light source does not emit light;
sequestering a portion of the carbon from the CO₂-rich source gas within the cyanobacteria through the process of photosynthesis to produce an exhaust stream, the exhaust stream having a reduced quantity of CO₂ as compared to the CO2-rich source gas; and
removing the exhaust stream from the containment area.

29. The method of claim 28, wherein the bottom of the trays define a plurality of holes such that the CO₂-rich source passing through the holes contacts the aquatic culture, the holes having an original average diameter.

30. The method of claim 28, further comprising the step of promoting circulation of the aquatic culture using a conveyance apparatus, the conveyance apparatus being in communication with the aquatic culture such that the aquatic culture moves with laminar flow.

31. The method of claim 28, wherein the conveyance apparatus comprises an Archimedes' screw.

32. The method of claim 28, wherein the conveyance apparatus comprises a reciprocal pump.

33. The method of claim 28, further comprising the step of recycling at least a portion of the exhaust gas into the containment area for further contact with the aquatic culture.

34. The method of claim 28, further comprising the step of capturing O₂ produced by the photosynthetic cyanobacteria in the exhaust stream.

35. The method of claim 28, further comprising the step of irradiating the aquatic culture with natural sunlight.

36. The method of claim 28, further comprising the step of filtering at least a portion of the cyanobacteria on the tray.

37. The method of claim 28, wherein circulation of the aquatic culture through at least a portion of the plurality of the trays is enhanced by the gravity through the portion of the plurality of trays being placed at an incline to the horizontal.

38. The method of claim 28, wherein at least a portion of the trays is made of a transparent material, facilitating irradiation by the light emitting diodes.

39. The method of claim 28, wherein the aquatic culture circulates through the trays at a speed of about less than 2 meters per second.

40. The method of claim 28, wherein at least a portion of the trays is lined with a non-stick material such that adhesion of cynobacteria to the trays is reduced.

41. The method of claim 28, wherein the original average diameter of the holes defined by the trays is less that an average diameter of the cyanobacteria.

42. The method of claim 28, wherein pressure within the containment area of the CO₂-rich source gas on the tray bottoms discourages the aqueous culture from flowing through holes defined by the trays.

43. The method of claim 28, wherein the tray further comprises a top such that the bottom, sides and top defines an enclosed tray area.

44. The method of claim 43, further comprising the step of controlling a gas pressure differential between the enclosed tray area and the containment area

45. The method of claim 43, wherein the gas pressure differential is controlled through the use of a pressure regulator.

46. The method of claim 28, wherein the gas velocity through the holes defined by the trays is less than about 40 meters per second.
